(19) United States
(12) Patent Application Publication
(10) Pub. No.: US 2010/0266682 A1
(43) Pub. Date: Oct. 21, 2010

(54) POLYETHYLENE GLYCOL-COATED SODIUM CARBONATE AS A PHARMACEUTICAL EXCIPIENT AND COMPOSITIONS PRODUCED FROM THE SAME

(76) Inventors: Nipun Davar, Pleasanton, CA (US); Pauly Kavalakatt, Fremont, CA (US); Indiran Pather, Rancho Cordova, CA (US); Sangita Ghosh, Foster City, CA (US)

Correspondence Address: O''Melveny & Myers LLP;IP&T Calendar Department LA-13-A7, 400 South Hope Street, Los Angeles, CA, 90071-2899, US

(21) Appl. No.: 12/764,489

(22) Filed: Apr. 21, 2010

Related U.S. Application Data
(63) Continuation-in-part of application No. 12/332,252, filed on Dec. 10, 2008.

(51) Int. Cl. A61K 9/14; A61K 31/437; A61K 9/20; A61K 9/48; A61P 25/20; A61K 31/46

(52) U.S. Cl. 424/451; 514/300; 424/490; 424/464; 514/291

(57) ABSTRACT

Non-effervescent pharmaceutical compositions having at least one particle of carbonate coated by a layer of polyethylene glycol that substantially covers the at least one carbonate particle are described. Compositions are also described where the compositions include a weakly basic therapeutic agent, a first pH-modifying agent having at least one particle of carbonate coated by a layer of polyethylene glycol, and a second pH-modifying agent. The weakly basic therapeutic agent could be, but is not limited to, zolpidem or scopolamine. Compositions including zolpidem and scopolamine are used to treat insomnia and depression, respectively.

### RELATED APPLICATION

This is a continuation-in-part of U.S. application Ser. No. 12/332,252, filed Dec. 10, 2008, which is expressly incorporated herein by reference in its entirety for all purposes.

### BACKGROUND OF THE INVENTION

Sodium carbonate is used in pharmaceutical compositions as an inactive ingredient. It is known that sodium carbonate shows instability that is manifested in various ways such as caking of sodium carbonate powder and hardening of pharmaceutical tablets containing sodium carbonate. Once powdered sodium carbonate cakes, it becomes less useful than free-flowing sodium carbonate as a pharmaceutical excipient due to processing and handling difficulties. Also, the hardening of pharmaceutical tablets affects the dissolution profile of the composition. Such hardening of tablets is believed to be triggered due to absorption of moisture by sodium carbonate upon exposure to humid conditions. Therefore, there is a need for a more stable form of sodium carbonate.

### BRIEF SUMMARY OF THE INVENTION

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug absorption is also affected by the properties of the drug. The ability of a molecule to pass through any mucous membrane is dependent upon its size, its lipid solubility, and the extent to which it is ionized, among other factors.

The extent to which a drug is ionized has further been investigated with respect to drug delivery across the mucous membranes. Ionization is dependent on the dissociation constant (pKa), and the pH of the molecule's surrounding environment. In its un-ionized form, a drug is sufficiently lipophilic to traverse a membrane via passive diffusion. In fact, according to the pH partition hypothesis, only un-ionized, non-polar drugs will penetrate a lipid membrane.

At equilibrium, the concentrations of the un-ionized form of the drug are equal on both sides of the membrane. As the concentration gradient drives passive diffusion, an increase in the percentage of the un-ionized form of a drug correspondingly increases the transmucosal absorption of the drug. Maximum absorption across the membrane is thought to occur when a drug is 100% in its un-ionized form. Similarly, absorption across the membrane decreases as the extent of ionization increases. Therefore, one may influence the extent of drug absorption across the mucous membranes of the oral cavity by altering the salivary pH.

In one embodiment, the present invention relates to stable excipients that aid in raising the pH of the saliva of a subject to increase the amount of drug that is absorbed across the mucous membranes of the oral cavity. The stable excipients include a carbonate particle or granule that is substantially covered or coated with polyethylene glycol.

In another embodiment, the present invention relates to a composition that includes polyethylene glycol (PEG)-coated granules of sodium carbonate characterized by a structure that has a layer of polyethylene glycol that substantially covers the granules of sodium carbonate.

In another embodiment, the present invention relates to a composition that includes PEG-coated granules of sodium carbonate characterized by a structure that has a lower rate of hydration compared to sodium carbonate.

In another embodiment, the present invention provides a composition having PEG-coated sodium carbonate manufactured by the following process: dissolving PEG in a solvent, spraying the PEG solution onto sodium carbonate particles, and drying the co-processed material to exclude the solvent. The co-processed sodium carbonate and PEG of the invention is in the form of a crystalline powder, which has an average particle size between about 100 microns and 1000 microns.

In another embodiment, the pharmaceutical compositions include PEG-coated sodium carbonate, along with other excipients, formed into a solid form, such as, a tablet, a compressed core, a disk, a lozenge, a bead, a slug, a film, a capsule, or a wafer. The pharmaceutical compositions comprising PEG-coated sodium carbonate result in no substantial change in the dissolution profile and no substantial change in the disintegration time under stress conditions of high temperature and relative humidity. These pharmaceutical compositions also resulted in no substantial increase in hardness under stress condition of high relative humidity.

In another embodiment, the present invention provides a pharmaceutical composition of a therapeutic agent, the composition includes (i) an effective amount of weakly basic therapeutic agent, (ii) a co-processed material consisting of sodium carbonate and polyethylene glycol and (iii) a second pH-modifying agent. The co-processed PEG-sodium carbonate and a second pH-modifying agent (sodium bicarbonate) are present in the composition in an amount sufficient to raise the pH of the saliva to at least 7.9.

In yet another embodiment, the pharmaceutical composition includes zolpidem, a first pH-modifying agent, and a second pH-modifying agent. The first pH-modifying agent includes at least one particle of carbonate coated by a layer of polyethylene glycol that substantially covers the at least one carbonate particle. The pharmaceutical composition could be a non-effervescent composition and would not contain an acid component. The second pH-modifying agent could be bicarbonate (such as sodium bicarbonate), sodium phosphate dibasic, potassium phosphate dibasic, sodium citrate, potassium citrate, sodium acetate, and sodium tartrate. After storage of this composition in an open dish at 30° C. and 65% relative humidity for at least two weeks, upon administration, the composition releases at least about 20%, alternatively at least about 25%, alternatively at least about 30%, alternatively at least about 35%, alternatively at least about 40%, alternatively at least about 50% of the zolpidem in a period of 5 minutes. Similarly, after storage at these conditions, the composition releases at least about 60%, alternatively at least about 65%, alternatively at least about 70%, alternatively at least about 75%, alternatively at least about 80% in a period of 10 minutes. The release testing was performed using methodology described in the United States Pharmacopoeia using the Type II apparatus with 500 ml of Simulated Intestinal Fluid as the dissolution medium at 25 RPM.

The pharmaceutical composition may include less than about 5 mg, alternatively less than about 4 mg, alternatively less than about 3 mg, alternatively less than about 2 mg, alternatively less than about 1 mg, alternatively about 4.5 mg, alternatively about 4 mg, alternatively about 3.75 mg, alternatively about 3.5 mg, alternatively about 3 mg, alternatively about 2.5 mg, alternatively about 2 mg, alternatively about 1.75 mg, alternatively about 1.5 mg, alternatively about 1.25 mg, alternatively about 1 mg, alternatively between about 0.25 to about 5 mg, alternatively between about 0.25 to about 4 mg, alternatively between about 0.25 to about 3 mg, alternatively between about 0.25 to about 2 mg, alternatively between about 1.5 to about 4.0 mg, alternatively between about 1.5 to about 3.75 mg of zolpidem hemitartrate or a molar equivalent of a pharmaceutically acceptable form of zolpidem.

In use, the pharmaceutical composition described above can be used for treating insomnia in a subject, e.g., middle of the night (MOTN) insomnia. The method of treatment includes the steps of administering to the subject a solid, non-effervescent pharmaceutical composition comprising zolpidem, a first pH-modifying agent and a second pH-modifying agent, where the first and second pH-modifying agents are present in an amount sufficient to raise the pH of the subject's saliva to a certain pH level. The amount of the first and second pH-modifying agents may be sufficient to raise the pH of the subject's saliva to about 7.9 or greater, about 8.0 or greater, about 8.1 or greater, about 8.2 or greater, about 8.3 or greater, about 8.4 or greater, about 8.5 or greater, about 8.6 or greater, about 8.7 or greater, about 8.8 or greater, about 8.9 or greater, about 9.0 or greater, about 9.1 or greater, about 9.2 or greater, about 9.3 or greater, about 9.4 or greater, about 9.5 or greater, about 9.6 or greater, about 9.7 or greater, about 9.8 or greater, about 9.9 or greater, about 10.0 or greater, about 10.1 or greater, about 10.2 or greater, about 10.3 or greater, about 10.4 or greater, or about 10.5 or greater, alternatively between about pH 8.0 to about pH 10.5, alternatively between about pH 8.0 to about pH 10.0, alternatively between about pH 8.5 to about pH 10.0, alternatively between about pH 8.5 to about pH 9.5. As mentioned above, the first pH-modifying agent includes at least one particle of carbonate coated by a layer of polyethylene glycol that substantially covers the at least one carbonate particle. Similarly, the second pH-modifying agent could be bicarbonate (such as sodium bicarbonate), sodium phosphate dibasic, potassium phosphate dibasic, sodium citrate, potassium citrate, sodium acetate, and sodium tartrate. The pharmaceutical composition can be administered intracavity, e.g., in the oral cavity. Administration includes but is not limited to, oral, sublingual, and buccal. Dosage amounts of zolpidem can include the amounts listed above.

In the case of MOTN insomnia, the composition could be administered to a subject who awakens from sleep and desires to resume sleep for less than 5 hours, i.e., the composition can be administered on an as needed basis after the subject has awakened rather than prophylactically (before the subject falls asleep). Methods of treating MOTN insomnia are described in U.S. application Ser. No. 11/439,873, published as US 2006-0281783, which is hereby expressly incorporated by reference in its entirety.

In another embodiment, the pharmaceutical composition includes scopolamine, a first pH-modifying agent, and a second pH-modifying agent. The first pH-modifying agent includes at least one particle of carbonate coated by a layer of polyethylene glycol that substantially covers the at least one carbonate particle. The second pH-modifying agent could be bicarbonate (such as sodium bicarbonate), sodium phosphate dibasic, potassium phosphate dibasic, sodium citrate, potassium citrate, sodium acetate, and sodium tartrate. The pharmaceutical composition may be a non-effervescent composition. Furthermore, the pharmaceutical composition may be a lozenge, disk, film, bead, wafer, compressed core, tablet, capsule, or powder formulation. The pharmaceutical composition may include less than about 10 mg, alternatively less than about 7.5 mg, alternatively less than about 5 mg, alternatively less than about 2.5 mg, alternatively less than about 1.0 mg, alternatively about 0.25 mg, alternatively about 0.5 mg, alternatively about 0.75 mg, alternatively about 1.0 mg, alternatively about 1.5 mg, alternatively about 2.0 mg, alternatively about 3.0 mg, alternatively about 4.0 mg, alternatively about 5.0 mg, alternatively about 6.0 mg, alternatively about 7.0 mg, alternatively between about 0.25 to about 10.0 mg, alternatively between about 0.25 to about 7.5 mg, alternatively between about 0.25 to about 5.0 mg, alternatively between about 0.25 to about 2.5 mg, alternatively between about 0.25 to about 1.75 mg, alternatively about 1.0 mg to 2.5 mg, alternatively about 1.3 mg to 2.2 mg, alternatively about 1.6 mg to 2.0 mg of scopolamine hydrobromide or a molar equivalent of a pharmaceutically acceptable form of scopolamine.

In use, the pharmaceutical composition described above can be used for treating depression in a subject. Scopolamine for use in treating depression was described in U.S. application Ser. No. 11/137,114, published as US 2006-0270698, which is hereby expressly incorporated by reference in its entirety. The method for treating depression includes the steps of administering to the subject the pharmaceutical composition comprising scopolamine, a first pH-modifying agent and a second pH-modifying agent, where the first and second pH-modifying agents are present in an amount sufficient to raise the pH of the subject's saliva to a certain pH level. The amount of the first and second pH-modifying agents may be sufficient to raise the pH of the subject's saliva to at least about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0, alternatively the pH of the subject's saliva is raised to between about pH 8.0 to about pH 10.0, alternatively between about pH 8.5 to about pH 10.0, alternatively between about pH 8.5 to about pH 9.5. As mentioned above, the first pH-modifying agent includes at least one particle of carbonate coated by a layer of polyethylene glycol that substantially covers the at least one carbonate particle. Similarly, the second pH-modifying agent could be bicarbonate (such as sodium bicarbonate), sodium phosphate dibasic, potassium phosphate dibasic, sodium citrate, potassium citrate, sodium acetate, and sodium tartrate. The pharmaceutical composition can be administered intracavity, e.g., in the oral cavity. Administration includes but is not limited to, oral, sublingual, and buccal. Dosage amounts of scopolamine can include the amounts listed above. The pharmaceutical composition could be administered 4 times a day (q.i.d.), alternatively 3 times a day (t.i.d.), alternatively 2 times a day (b.i.d.), alternatively once a day, alternatively once every 2, 3, 4, 5, 6, or 7 days.

In another embodiment, the pharmaceutical composition includes a weakly basic therapeutic agent, a first pH-modifying agent, and a second pH-modifying agent. The first pH-modifying agent includes at least one particle of carbonate coated by a layer of polyethylene glycol that substantially covers the at least one carbonate particle. The second pH-modifying agent could be bicarbonate (such as sodium bicarbonate), sodium phosphate dibasic, potassium phosphate dibasic, sodium citrate, potassium citrate, sodium acetate, and sodium tartrate. The pharmaceutical composition may be a non-effervescent composition that does not contain an acid component. The pharmaceutical composition may be a lozenge, disk, film, bead, wafer, compressed core, tablet, capsule, or powder formulation.

These compositions may be used to treat many diseases. The pharmaceutical composition can be administered intracavity, e.g., in the oral cavity. Administration includes but is not limited to, oral, sublingual, and buccal. The pharmaceutical composition can contain a sufficient amount of the first and second pH-modifying agents to raise the pH of the subject's saliva to above a certain pH level. This pH level can depend on the pKa of the weakly basic therapeutic agent. The first and second pH-modifying agents could be present in an amount sufficient to raise the pH of the saliva above the pKa of the weakly basic therapeutic agent, alternatively at least 0.5 pH units above the pKa, alternatively at least 1.0 pH units above the pKa, alternatively at least 1.5 pH units above the pKa, alternatively at least 2.0 pH units above the pKa. Alternatively, the first and second pH-modifying agents could be present in an amount sufficient to raise the pH of the saliva at least above 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, or 10.5. Similarly, the first and second pH-modifying agents could be present in an amount sufficient to raise the pH of the saliva at least about 7.8 to about 10.5, alternatively about 7.8 to about 10.0, alternatively about 8.0 to about 10.0, alternatively about 8.5 to about 10.0, alternatively about 9.0 to about 10.0.

The weakly basic therapeutic agent may be, but is not limited to, zolpidem, scopolamine, pilocarpine, ondansetron, granisetron, olanzapine, oxycodone, hydrocodone, hydromorphone, lincomycin, morphine, fentanyl, haloperidol, fluoxetine, prochlorperazine, carvedilol, pindolol, pentobarbital, pamaquine, methazolamide, methohexital, mercaptopurine, mepivacaine, meperidine, loxapine, idoxuridine, hydroflumethiazide, ketamine, erythromycin, flurazepam, amlodipine, gentamicin, buspirone, cimetidine, galanthamine, dextromethorphan, propranolol, timolol, nebivolol, labetalol, clonidine, tizanidine, ranitidine, pethidine, alphaprodine, tramadol, brompheniramine, mepyramine, acebutalol, amoxicillin, ampicillin, butabarbital, codeine, cyclopentolate, dantrolene, daunomycin, diazoxide, dibucaine, dimethylbarbituric acid, doxepin, droperidol, antazoline, azatadine, ketotifen, rivastigmine, tacrine, imipramine, risperidone, esmolol, phenyloin, mephenyloin, cyclobenzaprine, phenobarbital, ethosuximide, phensuximide, acetazolamide, noscapine, cyclizine, brompheniramine, endital, promethazine, atenolol, fenfluramine, norfloxacin, diphenhydramine, buprenorphine, hydroxyzine, naltrexone, chlorcyclizine, doxylamine, carbinoxamine, fluspirilene, naloxone, nalorphine, acebutolol, epirurubicin, daunorubicin, nadolol, sulfamerazine, sulfamethazine, penfluridole, bupivacaine, cyclosporine, domperidone, venlafaxine, amitriptyline, cisapride, fluvoxamine, sertraline, droxidopa, donepezil, memantine, pirlindole, mianserine, citalopram, clomipramine, nortriptyline, mirtazapine, procaine, terguride, clozapine, fluphenazine, perphenazine, thioridazine, trifluoperazine, mesoridazine, triflupromazine, clopenthixol, periciazine, or pipamazine.

In another embodiment, the invention includes a non-effervescent composition that includes a pH-modifying agent having at least one particle of carbonate coated by a layer of polyethylene glycol that substantially covers the at least one carbonate particle. The non-effervescent composition may further include an additional pH-modifying agent, which could be, but is not limited to, sodium bicarbonate, sodium phosphate dibasic, potassium phosphate dibasic, sodium citrate, potassium citrate, sodium acetate, or sodium tartrate. The non-effervescent composition could be in the form of a lozenge, disk, film, bead, wafer, compressed core, tablet, capsule, or powder formulation. The non-effervescent pharmaceutical composition can be administered intracavity, e.g., in the oral cavity, and could be administered, for example, orally, sublingually, and buccally. The non-effervescent pharmaceutical composition may further include a weakly basic therapeutic agent. Examples of weakly basic therapeutic agents include, but are not limited to, those listed above. The at least one particle of carbonate has a surface area. The layer of polyethylene glycol may cover at least about 50%, alternatively at least about 60%, alternatively at least about 70%, alternatively at least about 75%, alternatively at least about 80%, alternatively at least about 85%, alternatively at least about 90%, alternatively at least about 95% of the surface area of the particle.

In another embodiment, a layered composition includes carbonate and a pharmaceutically acceptable counter ion and polyethylene glycol such that the carbonate is coated by a layer of polyethylene glycol that substantially covers the carbonate. The carbonate could be in the form of a particle or a granule. The layer of polyethylene glycol could be about 0.1 to about 30 microns thick, alternatively about 0.1 to about 20 microns thick, alternatively about 0.1 to about 10 microns thick. The amount of polyethylene glycol coated on the carbonate may be about 4 to about 50%, alternatively about 4 to about 40%, alternatively about 10 to about 35% weight percent of carbonate and its counter-ion.

In another embodiment, a layered composition includes a granule of carbonate and a pharmaceutically acceptable counter ion and polyethylene glycol such that the polyethylene glycol is arranged in a layer that substantially covers the granule. The granule of carbonate has a surface area. The layer of polyethylene glycol may cover at least about 50%, alternatively at least about 60%, alternatively at least about 70%, alternatively at least about 75%, alternatively at least about 80%, alternatively at least about 85%, alternatively at least about 90%, alternatively at least about 95% of the surface area of the granule. The size of the granule may be from between about 1 to about 5000 microns, alternatively from between about 1 to about 4000 microns, alternatively from between about 1 to about 3000 microns, alternatively from between about 1 to about 2000 microns, alternatively from between about 500 to about 5000 microns, alternatively from between about 1000 to about 5000 microns, alternatively from between about 1500 to about 5000 microns, alternatively from between about 50 to about 1000 microns. The layer of polyethylene glycol may have the same thicknesses as described previously.

In another embodiment, a layered composition includes a particle including carbonate and a pharmaceutically acceptable counter ion and a layer of polyethylene glycol substantially surrounding the particle. A portion of the surface area of the particle may be covered by the polyethylene glycol as described above with respect to other embodiments. The particle may have a size as described previously with respect to other embodiments.

### BRIEF DESCRIPTION OF THE DRAWINGS

FIG. 1A is a diagram of batch fluid bed granulation (top spray). FIG. 1B is a diagram of batch fluid bed granulation (bottom spray).

FIG. 2 is a diagram of a high shear granulator.

FIG. 3 is a graph of the total hardness for various formulations at T=0 and after 1 day at 25° C. and 60% relative humidity.

FIG. 4 is a stereomicroscopy picture of granular PEG material.

FIG. 5 shows the granular PEG material of FIG. 4 under polarized light microscope.

FIG. 6 shows a stereomicroscopy picture of a granular PEG material.

FIG. 7 shows dissolution profiles of tablets at T=0 and at T=10 or 14 days at 30° C. and 65% relative humidity (open dish).

FIG. 8 shows dissolution profiles of the tablets for compositions at T=0 and at T=14 days at 30° C. and 65% relative humidity (open dish).

### DETAILED DESCRIPTION OF THE INVENTION

Others have tried to coat sodium carbonate in order to form a stable excipient. It is logical that coating of sodium carbonate with a material that is inert to moisture would prevent such moisture absorption and may enhance its stability and that of pharmaceutical compositions containing sodium carbonate. It is, however, unexpected that coating sodium carbonate with a hydrophilic polymer, such as polyethylene glycol, would retard moisture absorption or enhance stability of sodium carbonate. This invention arises in part from the unexpected enhancement of stability of sodium carbonate and pharmaceutical compositions containing granules or particles of sodium carbonate coated with a hydrophilic polymer, e.g., polyethylene glycol (PEG). The invention also arises in part from the unexpected observation of resistance to tablet hardening of sodium carbonate upon coating with polyethylene glycol.

Particles are a solid homogeneous substance. The size can range between about 1 and about 3000 microns, alternatively between about 100 and about 2500 microns, alternatively between about 500 and about 2500 microns, alternatively between about 500 and about 2000 microns.

Granules are a group of more than one particle. The size of granules can range from between about 1 and about 5000 microns, alternatively from between about 1 to about 4000 microns, alternatively from between about 1 to about 3000 microns, alternatively from between about 1 to about 2000 microns, alternatively from between about 500 to about 5000 microns, alternatively from between about 1000 to about 5000 microns, alternatively from between about 1500 to about 5000 microns.

This invention includes improved methods of administration of pharmaceutical compositions containing sodium carbonate in a solid dosage form wherein sodium carbonate granules and/or particles are coated with PEG.

The present invention provides an excipient that includes sodium carbonate and polyethylene glycol (PEG). Tablets containing the excipient have shown effective suppression of tablet hardening. The co-processed sodium carbonate and PEG of the invention is in the form of a crystalline powder, which has a particle size of about 50 to about 1000 microns, alternatively about 100 to about 750 microns, alternatively about 100 to about 500 microns, alternatively about 250 to about 500 microns. The crystalline powder has an average particle size of about 100 microns, alternatively about 200 microns, alternatively about 300 microns, alternatively about 350 microns, alternatively about 400 microns, alternatively about 500 microns. The thickness of the PEG coating ranges from about 0.1 to 30 microns, alternatively about 0.1 to about 20 microns, alternatively about 1 to about 20 microns, alternatively 1 to 10 microns, alternatively about 1 to about 5 microns.

In one embodiment, the present invention provides pharmaceutical compositions containing the PEG-coated sodium carbonate granules or particles, which are excipients that impart enhanced stability to the pharmaceutical compositions. Pharmaceutical compositions have been prepared previously using sodium carbonate or buffered soda (individual particles of sodium bicarbonate and sodium carbonate). (See, e.g., U.S. application Ser. No. 11/948,259, published as US 2008-0132535, which is hereby expressly incorporated by reference in its entirety.) Compositions containing PEG-coated sodium carbonate granules or particles show enhancement in stability as demonstrated by a dissolution profile that does not deteriorate or become slower over time.

### Process for Preparing PEG-Coated Sodium Carbonate

The present invention provides an excipient that includes sodium carbonate and PEG manufactured by a process that includes the steps of dissolving PEG in a solvent to form a PEG solution, coating the sodium carbonate by spraying the PEG solution onto the sodium carbonate, and drying the PEG-coated sodium carbonate. Alternatively, the excipient can be manufactured by a process that includes the steps of melting PEG, mixing the melted PEG with sodium carbonate, passing the mixture through a sieve or extruder, and then allowing the coated particles to cool. These processes can be performed by high shear granulation, fluid bed coating, melt extrusion, roller compaction, or melt coating. The manufacturing processes results in a coating of PEG on particles or granules of sodium carbonate. The amount of PEG coated on sodium carbonate can range in weight percent from about 4% to 50%, alternatively about 5 to 40%, alternatively about 5 to 30%, alternatively about 5 to 20%, alternatively about 5 to 10%.

### Characteristics of Components of PEG Solution

As mentioned above, the first step of a process for manufacturing the PEG-coated carbonate is to dissolve PEG in a solvent to form the PEG solution. The molecular weight of PEG that is dissolved in a solvent to form the PEG solution can range from about 200 to about 20,000 g/mol, alternatively about 1,450 to about 20,000 g/mol, alternatively about 1,000 to about 10,000 g/mol, alternatively about 1,450 to about 10,000 g/mol, alternatively about 1,000 to about 4,000 g/mol, alternatively about 1,450 to about 4,000 g/mol, alternatively about 1,000 to about 3,500 g/mol, alternatively about 1,450 to about 3,500 g/mol, and alternatively about 2,000 to about 3500 g/mol. The solvent used to form the PEG solution in the process described above can be water, organic solvents, or mixtures of organic solvents and water. The organic solvents may include, but are not limited to, alcohols of boiling point less than 100° C., tetrahydrofuran, acetone, ethyl acetate, methanol, ethanol, and isopropyl alcohol. If isopropyl alcohol is used in the solvent mixture to form the PEG solution, the range of isopropyl alcohol present in the solution may be about 10% to about 90%, alternatively about 30% to about 85%, alternatively about 50% to about 85%, alternatively about 60% to about 85%, alternatively about 70% to about 80%.

### Fluid Bed Coating

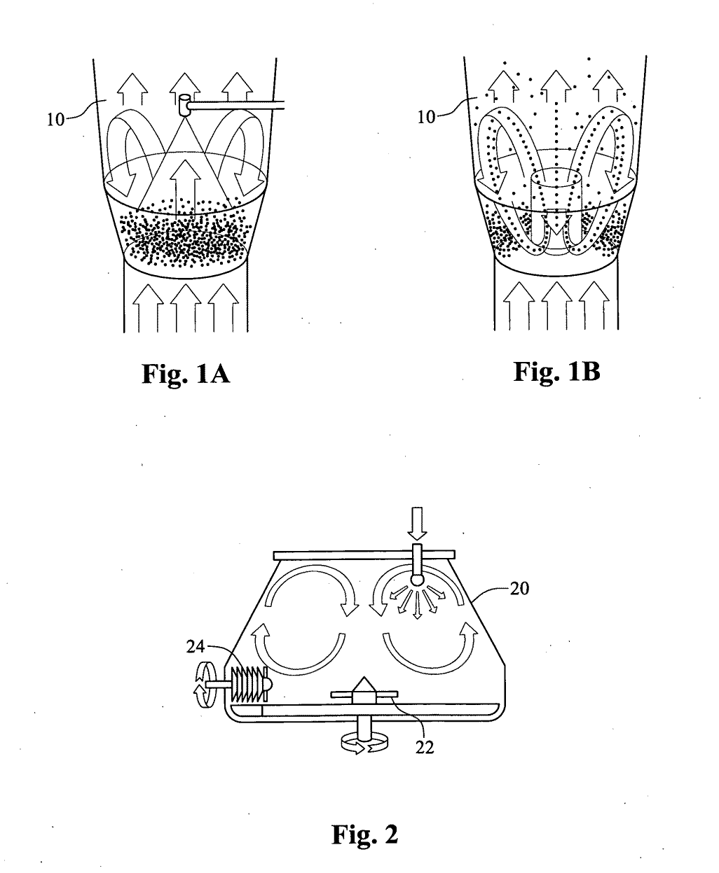

The diagrams in FIG. 1 illustrate batch fluid granulation with top spray (FIG. 1A) and bottom spray (FIG. 1B). Sodium carbonate is introduced into chamber 10 of the batch fluid bed granulator in powder form. The temperature of chamber 10 is then raised and air is introduced into chamber 10 through the powder bed to fluidize the powder. Simultaneously, the PEG solution is introduced into the chamber through a nozzle and sprayed onto sodium carbonate particles. After the PEG solution has been completely delivered, the fluidization (suspension of the particles in a rapidly moving stream of gas or vapor to induce flowing motion of the whole) continues to dry the PEG-coated granules and particles. The range of mixing time of PEG solution and sodium carbonate in the granulator is less than about 240 minutes, alternatively less than about 180 minutes, alternatively less than about 120 minutes, alternatively less than about 60 minutes, alternatively less than about 45 minutes. Alternatively, the mixing time of the PEG solution and sodium carbonate in the granulator can be about 20 to about 45 minutes, alternatively about 25 to about 40 minutes, alternatively about 30 to about 40 minutes. The PEG-coated carbonate product can then optionally be passed through a mesh of 10 to 30 microns to exclude very large particles.

After a particle or group of particles of sodium carbonate is substantially coated with the PEG solution, the material is dried. The range of drying temperature in the fluid bed dryer can be about 40° C. to 70° C., alternatively about 45° C. to 65° C., alternatively about 50° C. to about 60° C. The range of drying time in the fluid bed dryer can be less than about 120 minutes, alternatively less than about 60 minutes, alternatively less than about 15 minutes. Furthermore, the final material can be dried for about 10 hours to about 30 hours, alternatively about 18 hours to about 24 hours in a conventional oven.

### High Shear Granulation Method

For the high shear granulation method, PEG can be melted in a separate pot or dissolved in solvent as described above to form the PEG solvent. As seen in FIG. 2, the sodium carbonate can be placed in steel vessel 20 with mixer blade 22 and chopper blade 24. The PEG solution or melted PEG can be introduced into the jacketed vessel 20 containing sodium carbonate. During this process, the contents are mixed at high speed using mixer blade 22. Any wet clumps formed are reduced in size using chopper blade 24 located on the side of vessel 20. The wet mass can then be passed through a sieve and dried in a fluid bed dryer or in a conventional oven as described above.

### Melt Extrusion

For the melt extrusion method, PEG can be melted and mixed with sodium carbonate powder in a planetary or high shear mixer. Then PEG can be melted at a temperature range of about 50° C. to about 115° C., alternatively about 50° C. to about 80° C., alternatively about 55° C. to about 60° C. The blend is passed through a heated screen extruder (e.g., Luwa Corp) and collected on spheronizer. Granules are collected and dried in a conventional oven.

### Compositions

The present invention provides a pharmaceutical composition that includes (i) an effective amount of weakly basic therapeutic agent, (ii) a co-processed material consisting of sodium carbonate and polyethylene glycol, and (iii) a pH-modifying agent. The co-processed PEG-sodium carbonate and the pH-modifying agent are present in the composition in an amount sufficient to raise the pH of the saliva to at least above a certain pH depending on the pKa of the weakly basic therapeutic agent (see, e.g., TABLE 1). The PEG-sodium carbonate and the pH-modifying agent may be present in an amount sufficient, upon administration, to raise the pH of the subject's saliva to at least 0.5 pH units above the pKa, alternatively at least 1.0 pH units above the pKa, alternatively at least 1.5 pH units above the pKa, alternatively at least 2.0 pH units above the pKa of the weakly basic therapeutic agent. Alternatively, the PEG-sodium carbonate and the pH-modifying agent could be present in an amount sufficient to raise the pH of the saliva at least above 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0. Similarly, the PEG-sodium carbonate and the pH-modifying agent could be present in an amount sufficient to raise the pH of the saliva at least about 7.8 to about 11.0, alternatively about 7.8 to about 10.5, alternatively about 7.8 to about 10.0, alternatively about 8.0 to about 10.0, alternatively about 8.5 to about 10.0, alternatively about 9.0 to about 10.0.

The weakly basic therapeutic agents (or active ingredients) of the pharmaceutical composition can be, but are not limited to, zolpidem (and its pharmaceutically acceptable salts) and scopolamine (and its pharmaceutically acceptable salts).

In one aspect of the present invention, the active ingredient in the pharmaceutical composition is zolpidem hemitartrate or a pharmaceutically acceptable form of zolpidem. The composition may include less than about 10 mg of zolpidem hemitartrate or a molar equivalent amount of a pharmaceutically acceptable form of zolpidem, alternatively less than about 7.5 mg, alternatively less than about 5 mg, alternatively between about 1 mg to about 5 mg, alternatively between about 1.5 mg to about 4.5 mg, alternatively between about 1.75 mg and about 4 mg, alternatively between about 1.75 mg and about 3.5 mg, alternatively about 1.5 mg, alternatively about 1.75 mg, alternatively about 2 mg, alternatively about 2.5 mg, alternatively about 3 mg, alternatively about 3.5 mg, alternatively about 3.75 mg, alternatively about 4 mg, alternatively about 4.5 mg of zolpidem hemitartrate or a molar equivalent amount of a pharmaceutically acceptable form of zolpidem. Compositions that include zolpidem can be administered to subjects that suffer from insomnia. Zolpidem compositions and methods of treating various types of insomnia are described in, e.g., U.S. application Ser. No. 11/060,641, published as US 2005-226925, and U.S. application Ser. No. 11/439,873, published as US 2006-0281783, both of which are hereby expressly incorporated by reference in their entirety.

The weakly basic therapeutic agents (or active ingredients) of the pharmaceutical composition may also be scopolamine hydrobromide or a pharmaceutically acceptable form of scopolamine. The composition may include less than about 10 mg of scopolamine hydrobromide or a molar equivalent amount of a pharmaceutically acceptable form of scopolamine, alternatively less than about 7.5 mg, alternatively less than about 5 mg, alternatively between about 1 mg to about 5 mg, alternatively between about 1.5 mg to about 4.5 mg, alternatively between about 1.75 mg and about 4 mg, alternatively between about 1.75 mg and about 3.5 mg, alternatively about 1.5 mg, alternatively about 1.75 mg, alternatively about 2 mg, alternatively about 2.5 mg, alternatively about 3 mg, alternatively about 3.5 mg, alternatively about 3.75 mg, alternatively about 4 mg, alternatively about 4.5 mg of scopolamine hydrobromide or a molar equivalent amount of a pharmaceutically acceptable form of scopolamine. Compositions that include scopolamine can be administered to subjects that suffer from depression. Scopolamine for use in the treatment of depression and anxiety is described in, e.g., U.S. application Ser. No. 11/137,114, published as US 2006/0270698, which is hereby expressly incorporated by reference in its entirety.

TABLE 1 contains a list of weakly basic therapeutic agents, along with their pKa's, that can be used in accordance with this invention. These weakly basic therapeutic agents could be administered to subjects suffering from the indications listed below.

TABLE 1

| Drug | pKa | Indication |
|---|---|---|
| Zolpidem | 6.9 | Insomnia |
| Pilocarpine | 6.6 | Dry mouth including radiation-induced dry |
| | | mouth (xerostomia) and symptoms of dry |
| | | mouth in patients with Sjogrens syndrome |
| Scopolamine | 7.2 | Depression, excessive salivation, colicky |
| | | abdominal pain, bradycardia, sialorrhoea, |
| | | diverticulitis, irritable bowel syndrome, and |
| | | motion sickness |
| Ondansetron | 7.7 | Chemotherapy-induced nausea and |
| | | vomiting, substance abuse (including |
| | | alcohol abuse), obsessive-compulsive |
| | | disorder |
| Granisetron | 9.4 | Nausea and vomiting - e.g., associated with |
| | | initial and repeat courses of emetogenic |
| | | cancer therapy |
| Olanzapine | 7.3 | Schizophrenia, manic depression (bipolar |
| | | disorder), and substance abuse (including |
| | | alcohol abuse) |
| oxycodone | 8.9 | Pain management (moderate to severe) |
| hydrocodone | 8.9 | Pain management (moderate to severe) |
| hydromorphone | 8.2 | Pain management - e.g., pain due to |
| | | surgery, cancer, trauma/injury, burns, |
| | | myocardial infarction and colic |
| lincomycin | 7.5 | Bacterial infections - e.g., staphylococcal, |
| | | streptococcal, and Bacteroides fragilis |
| | | infections |
| Morphine | 7.9 | Pain management (moderate to severe) |
| Fentanyl | 8.5 | Pain management - e.g., treatment of cancer |
| | | patients with severe pain that breaks |
| | | through regular narcotic therapy |
| haloperidol | 8.3 | Schizophrenia (e.g. for patients who require |
| | | prolonged parenteral antipsychotic therapy) |
| | | Tourette's syndrome, and severe |
| | | hyperactivity |
| Fluoxetine | 8.7 | Major depressive disorder |
| prochlorperazine | 8.1 | Nausea and vomiting and management of |
| | | the manifestations of psychotic disorders |
| Carvedilol | 7.6 | heart failure of ischemic (mild or moderate) |
| | | or cardiomyopathic origin |
| Pindolol | 8.8 | Hypertension, edema, ventricular |
| | | tachycardias, and atrial fibrillation |
| Pentobarbital | 8.0 | Insomnia (e.g., short term treatment) |
| Pamaquine | 8.7 | |
| Methazolamide | 7.3 | Glaucoma (e.g., chronic open-angle |
| | | glaucoma and acute angle-closure |
| | | glaucoma) |
| Methohexital | 8.3 | Anesthetic |
| Mercaptopurine | 7.8 | Remission induction and maintenance |
| | | therapy of acute lymphatic leukemia |
| Mepivacaine | 7.6 | Analgesia and anesthesia |
| meperidine | 8.7 | Pain management (moderate to severe) |
| Loxapine | 6.6 | Psychosis (e.g., management of the |
| | | manifestations of psychotic disorders)s |
| Idoxuridine | 8.3 | keratoconjunctivitis and keratitis caused by |
| | | herpes simplex virus |
| hydroflumethiazide | 8.9 | Hypertension and edema (e.g., edema |
| | | associated with congestive heart failure, |
| | | hepatic cirrhosis, and corticosteroid and |
| | | estrogen therapy) |
| Ketamine | 7.5 | Anesthetic |
| erythromycin | 8.8 | Bacterial infections (e.g, treatment of |
| | | infections caused by susceptible strains of |
| | | microorganisms in the following diseases: |
| | | respiratory tract infections of mild to |
| | | moderate degree, pertussis, in the treatment |
| | | of infections due to Corynebacterium |
| | | minutissimum, intestinal amebiasis caused |
| | | by Entamoeba histolytica, acute pelvic |
| | | inflammatory disease caused by Neisseria |
| | | gonorrhoeae, skin and soft tissue infections |
| | | of mild to moderate severity caused by |
| | | Streptococcus pyogenes and |
| | | Staphylococcus aureus, syphilis, infections |
| | | caused by Chlamydia trachomatis, |
| | | nongonococcal urethritis caused by |
| | | Ureaplasma urealyticum, and Legionnaires' |
| | | disease caused by Legionella pneumophila) |
| flurazepam | 8.2 | Insomnia (e.g., short-term and intermittent |
| | | use in patients with recurring insomnia and |
| | | poor sleeping habits) |
| amlodipine | 8.6 | hypertension, chronic stable angina, and |
| | | vasospastic angina |
| gentamicin | 8.2 | Bacterial infections (e.g., treatment of |
| | | serious infections caused by susceptible |
| | | strains of the following microorganisms: P. aeruginosa, |
| | | Proteus species (indole-positive |
| | | and indole-negative), E. coli, Klebsiella- |
| | | Enterobactor-Serratia species, Citrobacter |
| | | species and Staphylococcus species |
| | | (coagulase-positive and coagulase- |
| | | negative)) |
| Buspirone | 7.2 | Anxiety disorders (e.g., short-term relief of |
| | | the symptoms of anxiety) and depression |
| | | (e.g., augmention of SSRI-treatment) |
| cimetidine | 6.98 | Acid-reflux disorders (GERD), peptic ulcer |
| | | disease, heartburn, and acid indigestion. |
| galanthamine | 8.32 | Dementia (e.g., mild to moderate of the |
| | | Alzheimer's type) |
| dextromethorphan | 8.3 | Dry cough |
| propranolol | 9.2 | Migraine (e.g., prophylaxis of migraine) |
| Timolol | 9.2 | High blood pressure and prevention of heart |
| | | attacks, prevention of migraine headaches, |
| | | treatment of glaucoma |
| Nebivolol | 8.6 | Hypertension |
| Labetalol | 8.7 | Hypertension |
| Clonidine | 8.05 | Hypertension, migraine, vascular headache. |
| | | and menopausal flushing |
| Tizanidine | 9.0 | Spasticity (e.g., management of increased |
| | | muscle tone associated with spasticity) |
| Ranitidine | 8.2 | Peptic ulcer disease (PUD), dyspepsia, |
| | | stress ulcer prophylaxis, and |
| | | gastroesophageal reflux disease (GERD) |
| Pethidine | 8.72 | Pain management (severe or constant) |
| alphaprodine | 8.73 | Pain management |
| Tramadol | 9.3 | Pain management (moderate or severe) |
| brompheniramine | 9.12 | common cold symptoms (including allergic |
| | | rhinitis, runny nose, itchy eyes, watery eyes, |
| | | and sneezing) |
| mepyramine | 8.9 | Disorders known to respond to |
| | | antihistamine therapy e.g. urticaria, rhinitis, |
| | | anaphylactic |
| acebutalol | 9.2 | Hypertension and ventricular premature |
| | | beats in adults |
| amoxicillin | 7.4 | Bacterial infections of the ear, nose, and |
| | | throat, the genitourinary tract, the skin |
| Ampicillin | 7.3 | Bacterial infections (e.g., respiratory, GI, |
| | | UTI and meningitis) due to E. coli, P. mirabilis, |
| | | enterococci, Shigella, S. typhosa |
| | | and other Salmonella, nonpenicillinase- |
| | | producing N. gononhoeae, H. influenzae, |
| | | staphylococci, streptococci) |
| butabarbital | 7.9 | Insomnia and anxiety disorders |
| Codeine | 7.9 | Pain management |
| cyclopentolate | 7.9 | Production of mydriasis and cycloplegia for |
| | | diagnostic purposes |
| dantrolene | 7.5 | Fulminant hypermetabolism of skeletal |
| | | muscle characteristic of malignant |
| | | hyperthermia crises; to prevent or attenuate |
| | | the development of clinical and laboratory |
| | | signs of malignant hyperthermia in |
| | | individuals judged to be malignant |
| | | hyperthermia susceptible |
| daunomycin | 8.2 | Remission induction in acute |
| | | nonlymphocytic leukemia (myelogenous, |
| | | monocytic, erythroid) of adults and for |
| | | remission induction in acute lymphocytic |
| | | leukemia of children and adults |
| Diazoxide | 8.5 | Hypertensive emergencies and |
| | | hypoglycemia secondary to insulinoma |
| Dibucaine | 8.5 | Anesthetic |
| Dimethylbarbituric acid | 7.1 | |
| Doxepin | 8.2 | Depression and/or anxiety (e.g., |
| | | psychoneurotic patients with depression |
| | | and/or anxiety) |
| droperidol | 7.6 | Nausea and vomiting (e.g., produce |
| | | tranquilization and to reduce the incidence |
| | | of nausea and vomiting in surgical and |
| | | diagnostic procedures) |
| antazoline | 7.2 | Conjunctivitis (e.g., hay-fever and allergic |
| | | conjunctivitis) |
| Azatadine | 9.3 | Symptoms of upper respiratory mucosal |
| | | congestion in perennial and allergic rhinitis, |
| | | and for the relief of nasal congestion and |
| | | eustachian tube congestion |
| Ketotifen | 8.75 | mild atopic asthma in children (e.g., as an |
| | | add-on or prophylactic oral medication in |
| | | the chronic treatment of mild atopic |
| | | asthmatic children) and prevention of |
| | | itching of the eye due to allergic |
| | | conjunctivitis (ophthalmic) |
| rivastigmine | 8.85 | Alzheimer's disease (mild to moderate) |
| Tacrine | 9.4 | Dementia (e.g., mild to moderate dementia |
| | | of the Alzheimer's type) |
| imipramine | 9.4 | depression and as temporary adjunctive |
| | | therapy in reducing enuresis in children |
| | | aged 6 years and older |
| risperidone | 7.9 | Schizophrenia, manic or mixed episodes of |
| | | bipolar I disorder, and obsessive compulsive |
| | | disorder |
| Esmolol | 9.5 | control of ventricular rate (e.g., in patients |
| | | with atrial fibrillation or atrial flutter in |
| | | perioperative, postoperative, or other |
| | | emergent circumstances where short term |
| | | control of ventricular rate with a short- |
| | | acting agent is desirable) and |
| | | noncompensatory sinus tachycardia (e.g., |
| | | where the rapid heart rate requires specific |
| | | intervention) |
| Phenytoin | 8.3 | Seizures (e.g., control of generalized tonic- |
| | | clonic and complex partial (psychomotor, |
| | | temporal lobe) seizures and prevention and |
| | | treatment of seizures occurring during or |
| | | following neurosurgery) |
| mephenytoin | 8.33 | Epilepsy (e.g., treatment of refractory |
| | | partial epilepsy) |
| cyclobenzaprine | 8.5 | Muscle spasm (e.g., adjunct to rest and |
| | | physical therapy for relief of muscle spasm |
| | | associated with acute, painful |
| | | musculoskeletal conditions) |
| phenobarbital | 7.4 | Seizures (e.g., all types of seizures except |
| | | absence seizures) |
| ethosuximide | 9.3 | Epilepsy |
| phensuximide | 9.2 | Epilepsy |
| Acetazolamide | 7.2 | Edema (e.g., due to congestive heart failure |
| | | and drug-induced edema); centrencephalic |
| | | epilepsies; and glaucoma (e.g., chronic |
| | | simple (open-angle)) |
| Noscapine | 7.8 | Cough (e.g., provides relief for the |
| | | symptoms of non-productive cough) |
| Cyclizine | 7.7 | Nausea, vomiting, and dizziness (e.g., |
| | | prevention and treatment of nausea, |
| | | vomiting, and dizziness associated with |
| | | motion sickness, and vertigo) |
| Brompheniramine | 9.12 | Symptoms of the common cold and allergic |
| | | rhinitis, such as runny nose, itchy eyes, |
| | | watery eyes, and sneezing |
| Endital | 7.5 | |
| promethazine | 9.1 | Allergic disorders, itching, nausea, and |
| | | vomiting |
| Atenolol | 9.5 | Hypertension and angina pectoris |
| fenfluramine | 9.1 | Obesity (e.g., exogenous obesity) |
| norfloxacin | 8.75 | Urinary tract infection |
| diphenhydramine | 8.3 | Symptoms associated with |
| | | Vertigo/Meniere's disease, nausea and |
| | | vomiting, motion sickness, and insect bites |
| Buprenorphine | 8.24 | Pain management, peri-operative analgesia |
| | | and opioid dependence |
| hydroxyzine | 7.1 | Symptomatic relief of anxiety and tension |
| | | associated with psychoneurosis and as an |
| | | adjunct in organic disease states in which |
| | | anxiety is manifested. Useful in the |
| | | management of pruritus due to allergic |
| | | conditions such as chronic urticaria |
| naltrexone | 8.13 | alcohol dependence and for the blockade of |
| | | the effects of exogenously administered |
| | | opioids |
| chlorcyclizine | 9.65 | |
| doxylamine | 9.2 | Insomnia |
| carbinoxamine | 8.1 | Symptomatic relief of seasonal and |
| | | perennial allergic rhinitis and vasomotor |
| | | rhinitis |
| fluspirilene | 7.32 | Schizophrenia |
| Naloxone | 7.82 | Depression (e.g., narcotic depression) |
| nalorphine | 7.59 | Cancer (e.g., adjuvant therapy in patients |
| | | with evidence of axillary node tumor |
| | | involvement following resection of primary |
| | | breast cancer) |
| acebutolol | 9.4 | |
| epirurubicin | 7.7 | |
| daunorubicin | 8.25 | Remission induction in acute |
| | | nonlymphocytic leukemia (myelogenous, |
| | | monocytic, erythroid) and in acute |
| | | lymphocytic leukemia |
| Nadolol | 9.67 | Cardiovascular disease (e.g., to treat |
| | | arrhythmias, angina pectoris, and |
| | | hypertension) |
| Sulfamerazine | 8.0 | Infections due to haemolytic streptococci, |
| | | meningococci, pneumococci, gonococci and |
| | | E. coli |
| Sulfamethazine | 7.4 | |
| penfluridol | 8.0 | Psychoses |
| Bupivacaine | 8.1 | Anesthesia or analgesia (e.g., for surgery, |
| | | for oral surgery procedures, for diagnostic |
| | | and therapeutic procedures, and for |
| | | obstetrical procedures) |
| cyclosporine | 8.99 | transplant rejection, rheumatoid arthritis, |
| | | and psoriasis |
| domperidone | 7.9 | Dyspepsia, heartburn, epigastric pain, |
| | | nausea, and vomiting |
| venlafaxine | 9.4 | Depression |
| amitriptyline | 9.4 | |
| Cisapride | 7.83 | Gastroesophageal reflux disease (e.g., |
| | | symptomatic treatment of adult patients |
| | | with nocturnal heartburn due to |
| | | gastroesophageal reflux disease) |
| fluvoxamine | 9.4 | Depression and obsessive compulsive |
| | | disorder (OCD) |
| Sertraline | 9.4 | Anxiety, bipolar disorders, and depression |
| Droxidopa | 7.88 | Parkinson's disease |
| Donepezil | 8.9 | Dementia |
| Memantine | 10.27 | Dementia |
| Pirlindole | 7.7 | Depression |
| Mianserine | 8.3 | Depression |
| Citalopram | 9.6 | Depression |
| Clomipramine | 9.4 | Depression |
| Nortriptyline | 9.7 | Depression |
| Mirtazapine | 7.7 | Depression (e.g., major depressive disorder) |
| Procaine | 8.9 | Anesthesia |
| Terguride | 7.2 | Hypertension (e.g., pulmonary arterial |
| | | hypertension) |
| Clozapine | 7.5 | Schizophrenia (e.g., management of |
| | | severely ill schizophrenic patients who fail |
| | | to respond adequately to standard drug |
| | | treatment for schizophrenia) |
| Fluphenazine | 7.9 | Psychotic disorders (e.g., management of |
| | | manifestations of psychotic disorders) |
| Perphenazine | 7.94 | Psychotic disorders (e.g., management of |
| | | the manifestations of psychotic disorders) |
| | | and for the control of severe nausea and |
| | | vomiting in adults |
| Thioridazine | 9.5 | Schizophrenia and anxiety (e.g., generalized |
| | | anxiety disorder) |
| Trifluoperazine | 8.1 | Anxiety disorders (e.g., depressive |
| | | symptoms secondary to anxiety and |
| | | agitation) |
| Mesoridazine | 8.2 | Schizophrenia, organic brain disorders, |
| | | alcoholism, and psychoneuroses |
| Triflupromazine | 9.2 | Psychoses and to control nausea and |
| | | vomiting |
| Clopenthixol | 7.6 | Schizophrenia (e.g., management of |
| | | manifestations of acute and chronic |
| | | schizophrenia) |
| Periciazine | 8.3 | Anxiety, psychoses, aggression, agitation, |
| | | impulsive behavior, and schizophrenia |
| Pipamazine | 8.6 | Nausea |

The pH-modifying agent may be, but is not limited to, sodium bicarbonate, sodium phosphate dibasic, potassium phosphate dibasic, sodium citrate, potassium citrate, sodium acetate, and sodium tartrate.

The pharmaceutical compositions may be, but are not limited to, lozenges, disks, films, heads, wafers, compressed cores, tablets, sustained release tablets, oral tablets, hard gelatin capsules, soft gelatin capsules and powder formulations. The pharmaceutical compositions may be administered intracavity—such as in the oral, nasal (intranasal), rectal, or vaginal cavities. In other words, the pharmaceutical compositions may be administered such that the drug is absorbed across the membranes of the cavity in which it is placed. For the oral cavity, the drug may be absorbed across any one or a combination of the following mucous membranes: the floor of the mouth (sublingual), the cheeks (buccal), the gums (gingival), the roof of the mouth (palatal), and the lining of the lips. Upon administration to the subject, the pH of the subject's saliva may be raised to at least 0.5 pH units above the pKa, alternatively at least 1.0 pH units above the pKa, alternatively at least 1.5 pH units above the pKa, alternatively at least 2.0 pH units above the pKa of the weakly basic therapeutic agent. Alternatively, the pH of the subject's saliva may be raised to at least above 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0. Similarly, the pH of the subject's saliva may be raised to at least about 7.8 to about 11.0, alternatively about 7.8 to about 10.5, alternatively about 7.8 to about 10.0, alternatively about 8.0 to about 10.0, alternatively about 8.5 to about 10.0, alternatively about 9.0 to about 10.0.

### Example 1

Comparison of tablet hardening propensity of sodium carbonate and PEG-coated sodium carbonate was determined by measuring hardness of tablets containing each of sodium carbonate, buffered soda (individual particles of sodium bicarbonate and sodium carbonate), or PEG-coated sodium carbonate. Blends containing a 1:1 mixture of sodium carbonate (Na₂CO₃), buffered soda, or PEG-coated sodium carbonate, each with anhydrous dicalcium phosphate (DCP), were compressed to hardness of about 3.7 kP. A tablet of DCP alone was used as a control. TABLE 2 indicates the composition of each tablet compared in the study. The tablets were exposed to 25° C. and 60% relative humidity in open Petri dishes for 1 day and the hardness and moisture content were measured.

TABLE 2

| | Calcium | | DCP-Buffered | DCP-10% PEG coated |
|---|---|---|---|---|
| | phosphate(DCP) | DCP-Na₂CO₃ | Soda | Na₂CO₃ |
| Ingredients | (% wt/mg) | (% wt/mg) | (% wt/mg) | (% wt/mg) |
| Na₂CO₃ | | 50/143.8 | | 45/133.2 |
| PEG-3350 | | | | 5/14.8 |
| Buffered Soda 43% | | | 50/142 | |
| Calcium | 99.5/274.6 | 49.5/142.3 | 49.5/140.6 | 49.5 |
| phosphate(DCP)(an) | | | | |
| Mg stearate | 0.5/1.4 | 0.5/1.4 | 0.5/1.4 | 0.5/1.5 |
| Total %/Tablet wt mg | 100/289 | 100/287.5 | 100/284 | 100/296 |

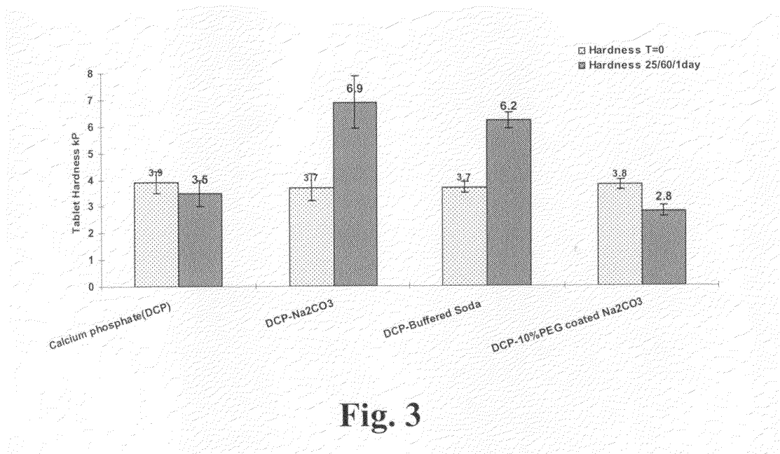

FIG. 3 shows the change in tablet hardness upon exposure to humidity after one day. Na₂CO₃ coated with 10 wt % PEG showed better resistance to change in hardness of the tablets upon exposure to 25° C. and 60% relative humidity for 1 day, with the hardness decreasing slightly from 3.8 kP to 2.8 kP. In comparison, tablets containing Na₂CO₃ or buffered soda showed an increase in hardness from 3.7 kP to 6.9 kP and 6.2 kP, respectively. Therefore, coating of sodium carbonate with PEG demonstrated an improvement over the two other forms of sodium carbonate in its tendency to resist tablet hardening.

### Example 2

### Preparation of PEG-Coated Sodium Carbonate

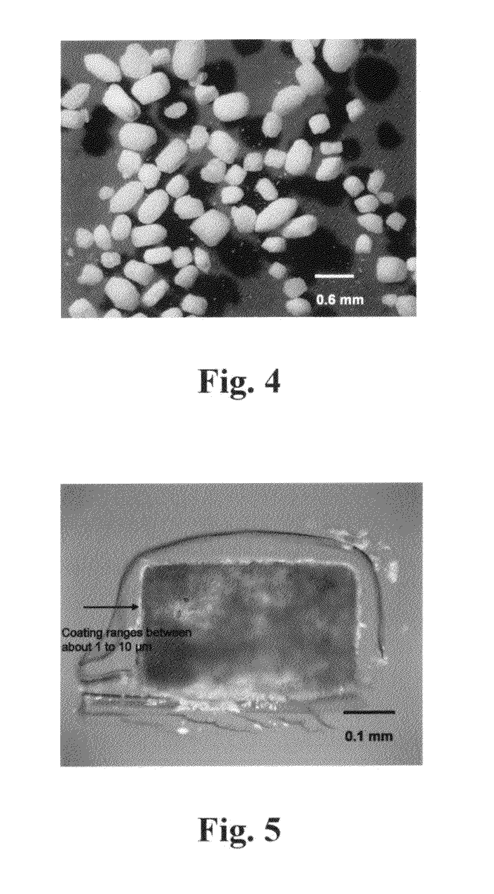

The coating liquid was prepared by dissolving 50 g of polyethylene glycol 3350 mol. wt (PEG 3350) in 200 ml of (80:20) isopropyl alcohol and water. The solution of PEG 3350 was sprayed on sodium carbonate (450 g) in a planetary mixer while mixing for 17 minutes. The resulting granulated material was sieved in a 20 mesh sieve and transferred to a steel tray and dried in an oven for 24 hours at 60° C. FIG. 4 shows a stereomicroscopy picture of the granular material obtained by the above-described process. FIG. 5 shows the granular material obtained by the above-described process under a polarized light microscope.

The final material was quantified for amount of PEG (as wt %) in the coating layer. The moisture content of the final material was determined for this purpose. Additionally, the amount of sodium carbonate was determined by titration. The following equations 1, 2 and 3 were used to calculate the extent of PEG coating.

$$\begin{matrix}\begin{matrix}Grams of {Na}_{2} {CO}_{3} \\ in Sample\end{matrix} = \begin{matrix}( mL Titrant \times {10}^{- 3} ) \times \\ ( Molarity of Titrant ) \times \\ ( 105.99 g \text{/} mol )\end{matrix} & Equation 1 \\ \begin{matrix}Grams of PEG \\ in Sample\end{matrix} = \begin{matrix}Weight of \\ Sample ( g )\end{matrix} - \begin{matrix}Amount of \\ {Na}_{2} {CO}_{3} in \\ Sample ( g )\end{matrix} - \begin{matrix}Amount of \\ Moisture in \\ Sample\end{matrix} & Equation 2 \\ Wt \% PEG = \frac{\begin{matrix}Grams of PEG in \\ Sample\end{matrix}}{\begin{matrix}Grams of \\ Sample\end{matrix}} \times 100 & Equation 3\end{matrix}$$

Results for the estimation of % PEG in the sample are shown in TABLE 3 below.

TABLE 3

| Weight of | mL of Titrant | Moisture | |
|---|---|---|---|
| Sample (g) | (0.998N HCl) | Content | Calculated Value (% PEG) |
| 1.894 | 16.3990 | 0.095% | 8.32% |

### Example 3

### Preparation of PEG-Coated Sodium Carbonate

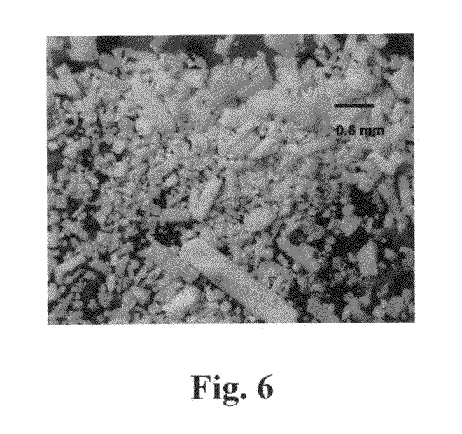

The coating liquid was prepared by dissolving 50 g of polyethylene glycol 3350 (PEG3350, mol. wt. 3350) in 200 ml of water. Sodium carbonate (450 g) was coated with the PEG 3350 solution in a bench top fluid bed granulator (FluidAir Model 002) using the bottom spray (Wurster coating) with further drying in the same granulator. The coating conditions used are detailed in TABLE 4 below. The coated particles were then discharged and sifted through a 20 mesh sieve. The final yield of PEG-coated sodium carbonate was 95.4%. FIG. 6 is a stereomicroscopy picture of the granular material obtained by the above-described process. The final material was quantified for amount of PEG (as wt %) in the coating layer as described above. Results for estimation of % PEG in the sample is shown in TABLE 5 below.

TABLE 4

| | |
|---|---|
| Inlet air temperature | 75° C.-80° C. |
| Outlet air temperature (during coating, record only) | 37° C. |
| Outlet air temperature (during drying, record only) | 48.5° C. |
| Atomization Air Pressure | 20 psi |
| Air flow | 25 SCFM |
| Solution spray rate | 9 ml/min |

TABLE 5

| Weight of | mL of Titrant | Moisture | |
|---|---|---|---|
| Sample (g) | (0.998N HCl) | Content | Calculated Value (% PEG) |
| 1.715 | 15.0908 | 0.095% | 6.83% |

### Example 4

Zolpidem lozenge compositions containing sodium carbonate and sodium bicarbonate were prepared according to the formulation set forth in TABLE 6.

TABLE 6

| | Quantity |
|---|---|
| Ingredients | (% wt) |
| Zolpidem tartrate | 1.67 |
| PEG 3350 | 0.8 |
| Na₂CO₃ | 8 |
| NaHCO₃ | 11 |
| flavor | 1.43 |
| color | 0.4 |
| sweetener | 0.7 |
| croscarmellose sodium | 5 |
| Silicon dioxide | 5 |
| Sodium stearyl fumarate | 5 |
| Pharmaburst | 61 |

A 3 kg blend was made according to the formulation in TABLE 6 in a V-shell blender (8 qt shell). A blending procedure was used involving step-wise sieving of all ingredients and blending them together in the V-shell blender. The blend was compressed at Pressima Kilian 8-station press. The compressed lozenges were then tested for appearance, hardness, weight, disintegration time, water content, and dissolution at 25 rpm. Physical attributes of pharmaceutical composition of zolpidem tartrate containing the PEG-coated granules and/or particles of sodium carbonate are listed in TABLE 7.

TABLE 9

Quantity

| Ingredients | Weight (mg) | % wt |
|---|---|---|
| Scopolamine hydrobromide | 0.5 | 1.43 |
| PEG 3350 | 0.28 | 0.8 |
| Na₂CO₃ | 2.52 | 7.2 |
| NaHCO₃ | 3.5 | 10 |
| peppermint flavor (oil) | 1.75 | 5.0 |
| Kollicoat ® IR | 23.299 | 66.57 |
| Sucralose | 0.35 | 1.0 |
| Glycerin | 2.8 | 8 |
| Total | 35 | 100.0 |

All excipients and scopolamine hydrobromide are weighed and dissolved in water and homogenized using a high speed homogenizer. The polymer solution is cast on a polyethylene casting liner at a wet thickness of about 2 to 4 mil and is dried in an oven. A clear glossy film is obtained after drying. The film is then equilibrated at room temperature for one day and then die cut into about 0.5 mg scopolamine hydrobromide unit doses.

### Example 7

### Preparation of Scopolamine Hydrobromide Lozenge

Scopolamine hydrobromide lozenge compositions are prepared according to the formulation set forth in TABLE 10.

TABLE 7
Zolpidem tartrate lozenges with PEG-Na₂CO₃

| Attributes | T = 0 |
|---|---|
| Hardness (kP) | 3.4 |
| Moisture Content | 0.34% |
| Disintegration time | 26 sec |
| Dissolution at 30 minutes | 96.85% |

### Example 5

Orally disintegrating lozenges were prepared with sodium carbonate and different second pH modifying agents as listed in TABLE 8. The pH of all of the lozenges in simulated saliva ranged between 9 and 10 and showed rapid disintegration times (DT) of 0.14 to 0.22 minutes.

TABLE 8

| | Ratio | Hardness | DT | pH |
|---|---|---|---|---|
| pH lowering agent | (Na₂CO₃:pH lowering agent) | (kP) | (min) | (2 ml simulated saliva) |
| Sodium Citrate | 1:1.4 | 3.5 | 0.17 | 9.75 |
| Sodium Phosphate dibasic | 1:1.8 | 4.3 | 0.14 | 9.64 |
| Sodium Tartrate dihydrate | 1:2.5 | 4.4 | 0.15 | 9.35 |
| Potassium phosphate dibasic | 1:2 | 5.8 | 0.22 | 9.75 |

### Example 6

### Preparation of Scopolamine Orally Dissolving Film

A single layer scopolamine film is produced according to the formulation set forth in TABLE 9.

TABLE 10

| Ingredient | mg/tab | % W/W | g/batch |
|---|---|---|---|
| Scopolamine HBr 3H₂O | 0.8 | 0.4 | 0.1 |
| PEG | 0.84 | 0.42 | 0.084 |
| Sodium carbonate | 7.64 | 3.82 | 0.764 |
| Sodium bicarbonate | 11.5 | 5.75 | 1.15 |
| Flavor | 3.0 | 1.5 | 0.3 |
| Color | 2.0 | 1.0 | 0.2 |
| Sucralose | 1.5 | 0.8 | 0.2 |
| Croscarmellose Na | 12.0 | 6.0 | 1.2 |
| Silicon dioxide | 4.0 | 2.0 | 0.4 |
| Sodium Stearyl fumarate | 10.0 | 5.0 | 1.0 |
| Pearlitol SD 200 | 146.8 | 73.4 | 14.68 |
| Total weight | 200.0 | 100.0 | 20.0 |

20 grams of the blend is made according to the formulation in TABLE 10 using a blending procedure as outlined in TABLE 11. Lozenges are compressed at Pressima Kilian 8-station press and the compressed lozenges tested for appearance, weight, pH, and disintegration time.

TABLE 11
Blending process for scopolamine hydrobromide lozenges

| Step No. | Blending Directions (40 gram batch) |
|---|---|
| 1 | Co-screen all of the following ingredients through #30 mesh |
| | and collect in a Teflon bottle |
| | Cabosil |
| | PEG-Na₂CO₃ |
| | sodium bicarbonate |
| | scopolamine hydrobromide |
| | flavor |
| | Sucralose |
| | Crosscarmellose Sodium |
| | SSF |
| | Pearlitol SD 200 (⅔ portion) |
| 2 | Co-screen the following through #60 mesh and collect in the |
| | Teflon bottle |
| | Color |
| | Pearlitol SD 200 (⅓ portion) |
| 3 | Blend the contents in the bottle for 10 minutes |

Physical attributes of pharmaceutical compositions of scopolamine hydrobromide containing the PEG-coated granules of sodium carbonate are listed in Table 12.

TABLE 12
Scopolamine hydrobromide lozenges with PEG-Na₂CO₃

| Attributes | T = 0 |
|---|---|
| Appearance | biconvex round tablets |
| Weight | 204 mg |
| pH | 9.6 |
| Disintegration time | 60 sec |

### Example 8

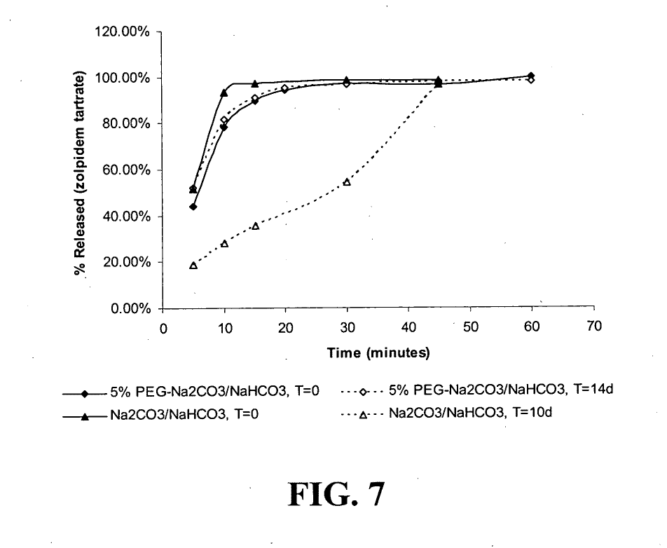

Stability studies for pharmaceutical compositions containing PEG-coated sodium carbonate and containing sodium carbonated and bicarbonate were performed. Two pharmaceutical compositions were prepared as shown in TABLE 13. Compressed tablets of each composition were stressed to study accelerated stability by placing the tablets in an open dish at 30° C. and 65% relative humidity. Physical attributes of tablets from each composition at an initial time (T=0) and after 10 or 14 days were measured (see TABLE 14). Dissolution profiles of tablets from each composition at an initial time (T=0) and at 10 or 14 days are shown in FIG. 7. An unchanged dissolution profile after 14 days at 30° C. and 65% relative humidity condition is unique to the zolpidem tartrate composition containing PEG-coated sodium carbonate. This same composition also maintains rapid disintegration up to 14 days (see TABLE 14a). The composition of zolpidem tartrate containing sodium carbonate shows slow dissolution in 10 days along with long disintegration time (TABLE 14b). As reported in TABLE 14, the percentage of moisture absorbed upon exposure to humidity by the tablet was higher with sodium carbonate (5.84%) compared to PEG-coated sodium carbonate (2.94%), which demonstrates that PEG acts as a barrier to external moisture.

TABLE 13

| | Lozenges | | Lozenges | |
|---|---|---|---|---|
| | (Na₂CO₃ and | | (PEG-Na₂CO₃ and | |
| | NaHCO₃ buffer) | | NaHCO₃ buffer) | |
| Ingredient | mg | % w/w | mg | % w/w |
| Zolpidem tartrate | 3.5 | 1.67 | 3.5 | 1.67 |
| Pharmaburst | 129.0 | 61.43 | — | — |
| Pearlitol SD 200 | — | — | 127.3 | 60.62 |
| Sodium carbonate | 17.0 | 8.10 | 17.0 | 8.10 |
| PEG3350 | — | — | 1.7 | 0.81 |
| Sodium bicarbonate | 23.0 | 10.95 | 23.0 | 10.95 |
| Croscarmellose sodium | 10.0 | 4.76 | 10.0 | 4.76 |
| Sodium stearyl fumarate | 10.0 | 4.76 | 10.0 | 4.76 |
| Silicon dioxide | 10.0 | 4.76 | 10 | 4.76 |
| Color | 2.0 | 0.95 | 2.0 | 0.95 |
| Flavor | 3.0 | 1.43 | 3.0 | 1.43 |
| Sucralose | 1.5 | 0.71 | 1.5 | 0.71 |
| Color | 1.0 | 0.48 | 1.0 | 0.48 |
| Total lozenge weight/Percent | 210.0 | 100 | 210.0 | 100 |

TABLE 14
(a) Zolpidem tartrate lozenges with PEG-Na₂CO₃ and NaHCO₃ buffer

| Attributes | T = 0 | T = 14 d |
|---|---|---|
| Hardness (kP) | 3.4 | 1.7 |
| Moisture Content | 0.34% | 2.94% |
| Disintegration time (sec) | 26 sec | 16 sec |
| Dissolution at 30 minutes | 96.85% | 96.8% |

(b) Zolpidem tartrate lozenges with Na₂CO₃ and NaHCO₃ buffer

| Attributes | T = 0 | T = 10 d |
|---|---|---|
| Hardness (kP) | 3.7 | 3.4 |
| Moisture Content | 1.46% | 5.84% |
| Disintegration time (sec) | 111 sec | 140 sec |
| Dissolution at 30 minutes | 98.5% | 54.4% |

### Example 9

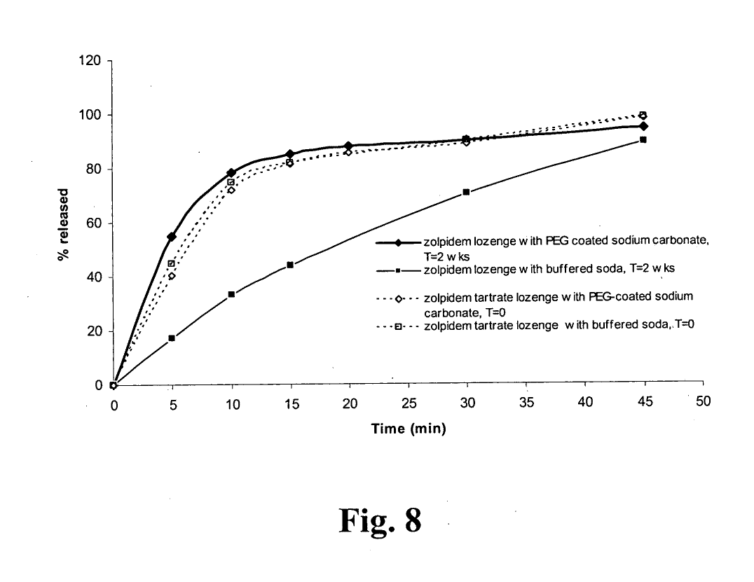

Stability studies for pharmaceutical compositions containing PEG-coated sodium carbonate and containing buffered soda were performed. Two pharmaceutical compositions of zolpidem tartrate were prepared as shown in TABLE 15 containing either PEG-coated sodium carbonate and sodium bicarbonate or buffered soda (single particle sodium carbonate and bicarbonate). Compressed tablets of each composition were stressed to study accelerated stability by placing in an open dish at 30° C. and 65% relative humidity. Dissolution profiles of the tablets from each composition at 14 days were determined as a measure of stability of the compositions and are reported in FIG. 8. An unchanged dissolution profile after 14 days at 30° C. and 65% relative humidity condition is unique to zolpidem tartrate composition containing PEG-coated sodium carbonate with sodium bicarbonate. The composition of zolpidem tartrate containing buffered soda shows slower dissolution in 14 days.

TABLE 15

| Zolpidem lozenge with | | | Zolpidem lozenge with | | |
|---|---|---|---|---|---|
| PEG-coated carbonate | | | buffered soda | | |
| Commodity Name | Mg/tab | % W/W | Commodity Name | Mg/tab | % W/W |
| Zolpidem Tartrate | 3.50 | 1.67 | Zolpidem Tartrate | 3.50 | 1.667 |
| Sodium carbonate | 17.0 | 8.1 | Buffered soda | 40.0 | 19.048 |
| Polyethyleneglycol-3350 | 1.7 | 0.81 | Spearmint flavor 913.004 | 3.0 | 1.429 |
| Sodium bicarbonate | 23.0 | 10.95 | Iron Oxide yellow | 1.0 | 0.476 |
| Spearmint flavor 913.004 | 3.0 | 1.43 | Sucralose | 1.5 | 0.714 |
| Color | 1.0 | 0.48 | Crosscarmellose sodium | 10.0 | 4.762 |
| Sucralose | 1.5 | 0.71 | Sylloid 244 FP | 8.0 | 3.810 |
| Crosscarmellose sodium | 10.0 | 4.76 | Silicon dioxide Cabosil | 2.0 | 0.952 |
| Silicon dioxide | 10 | 4.76 | Sodium Stearyl fumarate | 10.0 | 4.762 |
| Sodium Stearyl fumarate | 10.0 | 4.76 | Pharmaburst B2 | 131.0 | 62.381 |
| Pharmaburst B2 | 129.3 | 61.57 | | | |
| Total tablet weight | 210.0 | 100.00 | Total tablet weight | 210.0 | 100.00 |

Although the foregoing invention has, for the purposes of clarity and understanding, been described in some detail by way of illustration and example, it will be obvious that certain changes and modifications may be practiced which will still fall within the scope of the appended claims. It will also be understood that any feature or features from any one embodiment, or any reference cited herein, may be used with any combination of features from any other embodiment.

1. A pharmaceutical composition comprising:
zolpidem;
a first pH-modifying agent comprising at least one particle of a carbonate salt coated by a layer of polyethylene glycol that substantially covers the at least one carbonate salt particle; and
a second pH-modifying agent selected from the group consisting of sodium bicarbonate, sodium phosphate dibasic, potassium phosphate dibasic, sodium citrate, potassium citrate, sodium acetate, and sodium tartrate.

2. The pharmaceutical composition of claim 1, wherein the pharmaceutical composition is a non-effervescent composition.

3. The pharmaceutical composition of claim 1, wherein the pharmaceutical composition is selected from the group consisting of a lozenge, disk, film, bead, wafer, compressed core, tablet, capsule, and powder formulation.

4. The pharmaceutical composition of claim 1, wherein the zolpidem is present in amount of less than 10 mg of zolpidem hemitartrate or a molar equivalent amount of a pharmaceutically acceptable form of zolpidem.

5. The pharmaceutical composition of claim 1, wherein the second pH-modifying agent is sodium bicarbonate.

6. The pharmaceutical composition of claim 1, wherein, after storage in an open dish at 30° C. and 65% relative humidity for at least two weeks, upon administration, the composition releases at least 20% of the zolpidem in a period of 5 minutes.

7. The pharmaceutical composition of claim 1, wherein, after storage in an open dish at 30° C. and 65% relative humidity for at least two weeks, upon administration, the composition releases at least 30% of the zolpidem in a period of 5 minutes.

8. The pharmaceutical composition of claim 1, wherein, after storage in an open dish at 30° C. and 65% relative humidity for at least two weeks, upon administration, the composition releases at least 40% of the zolpidem in a period of 10 minutes.

9. The pharmaceutical composition of claim 1, wherein, after storage in an open dish at 30° C. and 65% relative humidity for at least two weeks, upon administration, the composition releases at least 60% of the zolpidem in a period of 10 minutes.

10. A method for treating insomnia in a subject, comprising the steps of:
administering to the subject a solid pharmaceutical composition comprising:
zolpidem;
a first pH-modifying agent comprising at least one particle of a carbonate salt coated by a layer of polyethylene glycol that substantially covers the carbonate salt particle; and
a second pH-modifying agent,
wherein the first and second pH-modifying agents are present in an amount sufficient to raise the pH of the subject's saliva to at least about 7.9.

11. The method of claim 10, wherein the solid pharmaceutical composition is a non-effervescent composition.

12. The method of claim 10, wherein the insomnia is middle-of-the-night insomnia.

13. The method of claim 10, wherein the composition is administered by a route selected from the group consisting of oral, sublingual, buccal, and intranasal.

14. The method of claim 10, wherein the composition is administered intracavity.

15. The method of claim 14, wherein the cavity is selected from the group consisting of oral, rectal, vaginal, and nasal.

16. The method of claim 10, wherein less than 10 mg of zolpidem hemitartrate or a molar equivalent amount of a pharmaceutically acceptable form of zolpidem is administered.

17. The method of claim 10, wherein the second pH-modifying agent is sodium bicarbonate.

18. The method of claim 10, wherein, after storage in an open dish at 30° C. and 65% relative humidity for at least two weeks, the composition releases at least 20% of the zolpidem in a period of 5 minutes following administration.

19. The method of claim 10, wherein, after storage in an open dish at 30° C. and 65% relative humidity for at least two weeks, the composition releases at least 40% of the zolpidem in a period of 10 minutes following administration.

20. A pharmaceutical composition comprising:
scopolamine;
a first pH-modifying agent comprising at least one particle of a carbonate salt coated by a layer of polyethylene glycol that substantially covers the at least one carbonate salt particle; and
a second pH-modifying agent.

21-25. (canceled)

26. A method for treating depression in a subject, comprising the steps of:
administering to the subject an orally dissolving film comprising:
scopolamine;
a first pH-modifying agent comprising a particle of a carbonate salt coated by a layer of polyethylene glycol that substantially covers the carbonate salt particle; and
a second pH-modifying agent,
wherein the first and second pH-modifying agents are present in an amount sufficient to raise the pH of the subject's saliva to at least about 8.8.

27-33. (canceled)

34. A pharmaceutical composition comprising:
a weakly basic therapeutic agent;
a first pH-modifying agent comprising at least one particle of a carbonate salt coated by a layer of polyethylene glycol that substantially covers the at least one carbonate salt particle; and
a second pH-modifying agent.

35-38. (canceled)

39. A non-effervescent pharmaceutical composition comprising a pH-modifying agent including at least one particle of a carbonate salt coated by a layer of polyethylene glycol that substantially covers the at least one carbonate salt particle.

40-49. (canceled)

50. A layered composition comprising:
carbonate and a pharmaceutically acceptable counter ion; and
polyethylene glycol,
wherein the carbonate and counter ion are coated by a layer of polyethylene glycol that substantially covers the carbonate and counter ion.

51-54. (canceled)

55. A layered composition comprising:
a granule comprising carbonate and a pharmaceutically acceptable counter ion; and
polyethylene glycol,
wherein the polyethylene glycol is arranged in a layer that substantially covers the granule.

56-63. (canceled)

64. A layered composition comprising:
a particle comprising carbonate and a pharmaceutically acceptable counter ion; and
a layer of polyethylene glycol substantially surrounding the particle.

65-70. (canceled)
